(19) United States
(12) Patent Application Publication
(10) Pub. No.: US 2004/0063726 A1
(43) Pub. Date: Apr. 1, 2004

(54) Methods and compounds for treating neurologic or neuropsychiatric disorders and identifying compounds to treat the same

(76) Inventor: Linda D Artman, Salt Lake City, UT (US)

Correspondence Address: TRASK BRITT, P.O. BOX 2550, SALT LAKE CITY, UT, 84110, US

(21) Appl. No.: 10/467,700

(22) PCT Filed: Feb. 8, 2002
(86) PCT No.: PCT/US02/03876

(51) Int. Cl. A61K 31/4965; A61K 31/255; A61K 31/12

(52) U.S. Cl. 514/255.06; 514/517; 514/675

(57) ABSTRACT

A method of treating a neurologic or neuropsychiatric disorder or disease in a mammal is provided. The method comprises administering a fatty acid amide hydrolase inhibitor in an amount sufficient to inhibit deamidation of a fatty acid amide. A method of identifying a fatty acid amide hydrolase inhibitor useful in the treatment of a neurologic or neuropsychiatric disorder is also provided. A method of identifying a fatty acid amide or fatty acid useful in the treatment of a neurologic or neuropsychiatric disorder is also provided.

### CROSS REFERENCE TO RELATED APPLICATIONS

This application claims the benefit of U.S. Provisional Patent Application Serial No. 60/267,310, filed Feb. 8, 2001 for METHODS AND COMPOUNDS FOR TREATING NEUROLOGIC OR NEUROPSYCHIATRIC DISORDERS AND IDENTIFYING COMPOUNDS TO TREAT THE SAME.

### TECHNICAL FIELD

The present invention relates to methods of treating neurologic or neuropsychiatric disorders and diseases, and more specifically to methods of inhibiting the metabolism and synthesis of fatty acids and fatty acid amides, and identifying pharmaceutically active compounds for treatment of neurologic or neuropsychiatric disorders and diseases.

### BACKGROUND ART

Fatty acids are essential to all organisms because they function as a source of energy, components of biomembranes, and metabolic regulators. More specifically, fatty acids (including short, long, saturated or unsaturated, and branched fatty acids) and their amides have been shown to have a wide variety of central nervous system mediated activities, including sedative, anticonvulsant, muscle relaxant, antinociceptive or analgesic, mood alteration, etc. ,

One such known use of exogenously administered fatty acids has application in the treatment of seizures and is referred to as a ketogenic diet (“KD”). The KD is a diet that is high in fatty acids (saturated and/or polyunsaturated depending on source) used primarily to treat children that are refractory to current anticonvulsant therapy. A large body of clinical and basic research literature exists demonstrating the efficacy of the KD. Specifically, the diet uses large amounts of vegetable oils, cream, butter, etc. to provide a high source of fatty acids and to produce an increase in the levels of ketone bodies, such as β-hydroxybutyrate and acetoacetate. These ketone bodies, however, have not been found to possess remarkable anticonvulsant activity nor are diets that produce higher levels of ketosis necessarily more anticonvulsant in nature. With the KD, the fatty acid metabolism is altered or, stated differently, the body is forced to derive its energy from the breakdown of fatty acids. This effect is similar to that observed in a fasting state, which is another historically known treatment for seizures. The KD has also shown anticonvulsant effect in animal models, appearing to closely mimic the ketogenic state achieved in humans. The diet, while efficacious in children, has not been a proven or clinically established therapy in adult epilepsy, mainly due to the lack of diet control by adult patients. Due to the difficulty in maintaining the diet, patients and medical practitioners are often reluctant to use the diet as a first line therapy.

A family of dual-action enzymes known as fatty acid amide hydrolases (“FAAH”) (also known as synthases or amidases) is responsible for the deamidation of fatty acid amides (“FAA”) to their corresponding acids, as well as for the synthesis of fatty acids to their corresponding amides, according to the following exemplary illustration:

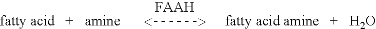

Although the foregoing reactions are substrate driven, the process is favored towards the production of the fatty acids. Amides of fatty acids historically have more potent therapeutic activities than their corresponding fatty acids but have proven to be short lived entities that are readily converted to their corresponding fatty acids. However, specific substituents on some short, branched chain FAAs have led to FAAs that are more resistant to hydrolysis. FAAHs are known to be widely distributed throughout the CNS and one particular member of the FAAH family has considerable overlap with cannabinoid receptors (e.g., CB1). Fatty acids (including short, long, and branched) and their amides have been shown to have a wide variety of central nervous system mediated activities, including sedative, anticonvulsant, muscle relaxant, antinociceptive or analgesic, mood alteration, etc.

Other treatments for seizures include use of specific fatty acids as first line therapy. For example, valproic acid (“VPA”) (available as Depakene® from Abbott) is a well-known anticonvulsant pharmaceutical. Valproic acid is a substrate for CoA pathways. However, once a valproylCoA molecule is formed, the new molecule no longer acts as a substrate for further pathways and, thus, the body must find alternative pathways to eliminate valproic acid and valproylCoA. VPA has been shown to alter fatty acid synthesis in the body and studies have reported that when severe ketosis is present, it can elicit side effects, such as hepatotoxicity. Valpromide (“VPD”), the corresponding amide of VPA, has also demonstrated a range of therapeutic activities in humans. However, like many amides of fatty acids, such as anandamide (a product of arachidonic acid and ethanolamine), VPD is readily converted to its corresponding fatty acid.

Isovaleric acid (“IVA”) is an endogenous fatty acid resulting from the breakdown of leucine and fatty acids in the body. Isovaleramide (“IVM”), the amide of IVA is also an endogenous amide that has demonstrated anticonvulsant effects. IVA is a normal high affinity substrate for acetyl CoA pathways and is the major route of subsequent metabolism for IVM. IVM does not appear from initial studies to be a substrate for CoA and must be converted to IVA or other branched acid before it can become a substrate for the CoA pathways. As with IVM, numerous other endogenous bioactive amides, such as anandamide, are hydrolyzed or inactivated by FAAHs and converted to less active or inactive deamidated fatty acids.

Thus, there exists a need in the art for methods to inhibit the hydrolysis of presently unidentified FAAs to their corresponding acids by inhibition of FAAHs (possibly other members of the family of FAAHs that have as yet to be identified or characterized) in order to improve the therapeutic action of fatty acid amides in a number of maladies, such as epilepsy, mood disorders, sleep disorders, restlessness syndromes, migraine headaches, movement disorders, spasticity, pain disorders, anxiety, and neurodegenerative disorders. There also exists a need in the art of increasing levels of endogenous and exogenously administered FAAs as a means of treating the aforementioned maladies.

### DISCLOSURE OF INVENTION

The present invention relates to a method of treating neurologic or neuropsychiatric disorders in a mammal which includes administering a FAAH inhibitor to a mammal (e.g., a human) in an amount sufficient to inhibit deamidation of fatty acid amides, as previously described herein. In a more specific embodiment, a method for altering the levels of fatty acid amides in a mammal is described which includes administering to a mammal a FAAH inhibitor in order to inhibit the conversion of FAA to a fatty acid and, in turn, treat neurologic or neuropsychiatric disorders, such as epilepsy.

Another method of the invention relates to inhibiting the breakdown of exogenously administered fatty acid amides in mammals believed to be suffering from neurologic or neuropsychiatric disorders. The method includes administering a dietary source of fatty acid and a FAAH inhibitor to the mammal being treated in order to inhibit the reversible conversion between FAAs and fatty acids, and thus, increase the concentration of FAAs in the body, to enhance or mimic one possible effect of a ketogenic diet.

Yet another method of the invention relates to a method of treating epilepsy, a mood disorder, a migraine headache, a spastic condition, a restless limb syndrome, or a movement disorder by administering a FAAH inhibitor that inhibits the deamidation of cannabamimetic FAAs, such as anandamide and oleamide.

The present invention additionally relates to methods for identifying fatty acids, FAAs, and FAAH inhibitors that are useful in the treatment of neurologic or neuropsychiatric disorders. One such method includes providing at least one known FAA and/or fatty acid or fatty acid diet (e.g., ketogenic diet), and contacting the FAA(s) and/or fatty acid(s) with FAAH inhibitor to screen for FAAH activity. The concentrations of fatty acid amide and corresponding fatty acid present or available are then measured, the fatty acid amide(s) or fatty acid(s) are administered to a mammal, and the ability of the fatty acid amide, fatty acid, and/or FAAH inhibitor to treat the neurologic or neuropsychiatric disorder is assayed.

Another embodiment of the invention relates to pharmaceutical compositions of any biologically or pharmaceutically active fatty acid(s), FAA(s), FAAH inhibitors, or derivatives thereof that are disclosed in the aforementioned method for identifying such compounds. Pharmaceutical compositions of fatty acids, FAAs and FAAH inhibitors, or derivatives of any of these compounds may alternatively be selected from those biologically or pharmaceutically active compounds that are derived from the administration of the KD. Alternatively, precursors of biologically active fatty acid(s), FAA(s), and FAAH inhibitors may be administered to a mammal, which bypasses the need to manage a diet to produce these bioactive compounds and provides a more controlled source of the biologically active compounds.

A particular embodiment of the invention relates to a method of identifying novel FAAHs by administering isovaleramide to a mammal and monitoring the activity of FAAHs and products resulting from the metabolism of the administered isovaleramide. Alternatively, FAAHs may be employed to screen for novel FAAH inhibitors or to develop novel FAAs that are more resistant to hydrolysis and which retain their biological activity for extended periods. Alternatively, a method of identifying novel, biologically active fatty acids and FAAs includes administering a fatty acid diet (e.g. ketogenic diet) to a mammal, identifying novel fatty acids and FAAs resulting from the metabolism of the administered dietary fatty acids, and assaying or measuring the ability of the resulting fatty acids and FAAs to treat neurologic or neuropsychiatric disorders or diseases. In one particular aspect of the method, the resulting fatty acids and FAAs are altered by contacting the same with FAAH inhibitors to produce novel fatty acids and FAAs. In an alternative embodiment, novel diets of selected fatty acids and/or FAAs may be formulated for administration to mammals in order to treat neurologic or neuropsychiatric disorders or diseases.

The present invention also includes a method for identifying fatty acid amides and FAAH inhibitors useful in the treatment of neurologic or neuropsychiatric disorders. According to this aspect of the invention, at least one fatty acid amide or fatty acid is provided and brought into contact with FAAH inhibitor. The resulting concentrations of fatty acid amide and corresponding fatty acid are measured and then the fatty acid amide or fatty acid is administered to a mammal. The ability of said fatty acid amide, fatty acid, and/or FAAH inhibitor to treat a particular neurologic or neuropsychiatric disorder is then analyzed.

### BRIEF DESCRIPTION

The term “mammal” is intended to mean, for the purpose of this invention, humans, domestic animals, farm animals, wild animals, or any other vertebrate the females of which have milk-secreting glands for feeding their offspring.

The terms “treating” and “treatment” are intended to mean the amelioration or complete elimination of the symptoms of the disorder and/or the pathogenic origin of the disorder, as part of therapeutic or prophylactic therapy. A compound uncovered by the screening method is characterized as having potential therapeutic utility in treatment because clinical tests have not yet been conducted to determine actual therapeutic utility.

The term “administer” is intended to mean that any method of treating a subject with a substance, such as orally, intravenously, nasally, intramuscularly, subcutaneously, topically, rectally, or via inhalation therapy.

The term “neurologic or neuropsychiatric disorder or disease” means a disorder or disease of the nervous system including, but not limited to, epilepsy, pain, anxiety, sleep disorders, mood disorders, migraine headaches, spastic conditions, restless limb syndromes, movement disorders and neurodegenerative diseases. Also meant by “neurologic or neuropsychiatric disorder or disease” are those disease states and conditions in which a neuroprotectant, anticonvulsant, anxiolytic, analgesic, muscle relaxant and/or adjunct in general anesthesia may be indicated, useful, recommended or prescribed.

The term “movement disorder” includes a neurophysiological movement disorder that adversely affects an individual's motor skills. Symptoms associated with these disorders include tremors, disorders and slowness of movement, difficulties with balance, and an inability to coordinate three dimensional hand movements. The disorders also cause rigidity (an inability to start and plan a rapid movement), bradykinesia (an inability to reach a high terminal velocity), festination (an inability to decelerate), and dystonia (an inability to smoothly control a movement of two muscles in a specific direction). Movement disorders include, but are not limited to, Parkinson's disease, parkinsonism, drug induced parkinsonism, Parkinson's plus syndromes, Wilson's disease, supranuclear paresis, Shy Drager disease, Lewy body disease, degenerative diseases of the basal ganglia, and poisonings (e.g. by manganese, carbon monoxide, carbon disulfide, hypercalcemia, drug ingestion with MPTP or MPP+ and azide overdose).

The term “neurodegenerative disease” includes, but is not limited to, cerebral cortical atrophy, Lou Gehrig's disease, multiple sclerosis, Lewy-body dementia, Pick disease, Alzheimer's disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstman-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and ophthalmoplegia.

The term “anticonvulsant” means a compound capable of reducing convulsions produced by conditions such as simple partial seizures, complex partial seizures, status epilepticus, and trauma-induced seizures such as occur following head injury, including head surgery.

The terms “therapeutic dose” or “sufficient amount” mean an amount of a compound that relieves to some extent one or more symptoms of the disease or condition of the patient. Additionally, by these terms is meant an amount that returns to normal, either partially or completely, physiological or biochemical parameters associated or causative of the disease or condition.

The term “pharmaceutical composition” means a therapeutically effective amount of a compound of the present invention in a pharmaceutically acceptable carrier, i.e., a formulation to which the compound may be added to dissolve or otherwise facilitate administration of the compound. Examples of pharmaceutically acceptable carriers include water, saline, and physiologically buffered saline.

### BEST MODE OR MODES FOR CARRYING OUT THE INVENTION

A particular embodiment of the method involves treating neurologic or neuropsychiatric disorders in a mammal by administering a FAAH inhibitor to a mammal in an amount sufficient to inhibit deamidation of fatty acid amides to their corresponding fatty acids or synthesis of fatty acids to their corresponding fatty acid amides. Neurologic or neuropsychiatric disorders that are known to be specifically affected by fatty acids, ketosis, and fatty acid amides, and for which the method of the invention is particularly suited, include epilepsy, pain, sleep disorders, mood disorders, migraine headaches, spastic conditions, restless limb syndrome, anxiety, neurodegenerative diseases, and movement disorders. Administration of the FAAH inhibitors is equally effective to inhibit deamidation/synthesis of endogenous fatty acid amides and fatty acids, as well as the deamidation/synthesis of exogenously administered FAAs and their respective fatty acids. The amount of FAAH inhibitor administered is sufficient to inhibit deamidation of endogenous fatty acid amides and exogenously administered fatty acid amides.

In a particular embodiment of the method, preferred FAAH inhibitors for use with the present invention include FAAH inhibitors that inhibit the deamidation of cannabamimetic FAAs, and more preferably, those FAAH inhibitors that inhibit deamidation/synthesis of anandamide and oleamide to their respective fatty acid counterparts, arachidonate and oleic acid. These preferred FAAH inhibitors are particularly useful as agents for treatment of epilepsy, mood disorders, migraine headaches, spastic conditions, restless limb syndrome, or movement disorders. More specifically, FAAH inhibitors suitable for use with the present invention include any known FAAH inhibitor, such as palmitylsulfonyl fluoride, stearylsulfonyl fluoride, 2-octyl-γ-bromoacetoacetate, phenylmethylsulfonyl fluoride, trifluoromethyl ketones, diazomethylarachidonyl ketone, and pyrazinamide.

The method of the invention is also effective as a therapeutic treatment for a variety of neurologic or neuropsychiatric disorders that are directly or indirectly affected by serotonin, such as mood disorders and motor functions. Thus, in another embodiment of the method, the FAA inhibitor may be selected to inhibit the deamidation of a fatty acid amide that modulates serotonergic neurotransmissions.

Alternatively, the aforementioned methods may also further include administration of exogenous fatty acids or FAAs to a mammal in order to alter levels of fatty acids and their amides having neuromodulatory properties in order to treat neurologic or neuropsychiatric disorders or diseases. Another embodiment of the method further includes administration of one or more ketogenic amino acids to the mammal, such as, for example, leucine, lysine, phenylalanine, tyrosine, and tryptophan. It is understood that the FAAH inhibitors of the present invention may also be administered in conjunction with pharmaceutical compositions intended for treatment of neurologic or neuropsychiatric disorders or diseases, such as, for example, administering the FAAH inhibitors of the present invention in conjunction with anticonvulsants.

In a more specific embodiment, a method for altering the levels of FAAs in a mammal is described which includes administering to the mammal a FAAH inhibitor in order to inhibit the conversion of the FAA to a fatty acid and, in turn, treat neurologic or neuropsychiatric diseases and disorders, such as epilepsy. Since the conversion from FAA to fatty acid is reversible, the ability of a FAAH inhibitor to inhibit the conversion from fatty acid to FAA is also included in the scope of the invention.

Yet another method of the invention relates to inhibiting the breakdown of exogenously administered FAAs in mammals believed to be suffering from neurologic or neuropsychiatric diseases or disorders. The method includes administering to the mammal a dietary source of a fatty acid or fatty acid amide. A FAAH inhibitor is also administered to the animal being treated in order to inhibit the reversible conversion between fatty acid amides and fatty acids, and thus, increase the concentration of fatty acids and/or FAAs in the body, alter fatty acid metabolism, produce ketosis, and enhance the effect of a ketogenic diet.

The present invention additionally relates to methods for identifying FAAs, fatty acids, FAAHs, and FAAH inhibitors that are useful in the treatment of neurologic or neuropsychiatric disorders. One such method includes providing at least one FAA and/or fatty acid, and contacting the FAA(s) and/or fatty acid(s) with a FAAH inhibitor. The concentrations of fatty acid amide and corresponding fatty acid present or available are then measured, the FAA(s) or fatty acid(s) are administered to a mammal, and the ability of the FAA, fatty acid, and/or FAAH inhibitor to treat the neurologic or neuropsychiatric disorder is assayed. This method has particular utility in the identification of an active moiety or discovery of a mechanism of action of fatty acids, FAAs, or other pharmaceutical compounds (e.g., valproic acid, valpromide, or isovaleramide) which have been identified as active in the treatment of neurologic or neuropsychiatric disorders or diseases such as depression, pain, spasticity, migraines, mood disorders, and dysthymic disorders.

In a more specific embodiment, the present invention relates to a method for identifying FAAH inhibitors that are useful in the treatment of neurologic or neuropsychiatric disorders. The method comprises providing a FAAH and at least one FAA or fatty acid. The FAAH may include, but is not limited to, oleamide hydrolase or anandamide amidase. The FAAH may be commercially obtained or cloned into an expression vector and recombinantly expressed, as described in U.S. Pat. No. 6,271,015 (the '015 patent), the contents of which are hereby incorporated by reference. The FAAH may be substantially pure or may be a fraction isolated from a membrane preparation, as described in the '015 patent and Edgemond et al., Synthesis and Characterization of Diazomethylarachidonyl Ketone: An Irreversible Inhibitor of N-Arachidonylethanolamine Amdiohydrolase, The Journal of Pharmacology and Experimental Therapeutics, 286(1): 184-190, the contents of which are hereby incorporated by reference. The necessary purity of the FAAH may depend on the assay used to determine potency of the FAAH inhibitor, discussed below.

The fatty acid may be purchased from a chemical supply company, such as Sigma-Aldrich Co. (St. Louis, Mo.). Fatty acids used in the present invention may include a straight or branched alkane chain of varying length, such as for example, a length of up to approximately 24 carbons. Preferably, a chain length of up to approximately 15 carbons is used. Specifically, a chain length of approximately 6-15 carbons is used. More specifically, a chain length of 3-6 carbons is used. However, it is contemplated that the alkane chain may include more than 24 carbons. In addition, the fatty acid may include at least one olefin in the carbon chain. The location, number, and cis or trans stereochemistry of the olefin may be varied to produce a diverse number of fatty acids for use in the present invention

FAAs used in the present invention may include a straight or branched alkane chain of varying length, such as for example, a length of up to approximately 24 carbons. Preferably, a chain length of up to approximately 15 carbons is used. Specifically, a chain length of approximately 6-15 carbons is used. More specifically, a chain length of 3-6 carbons is used. However, it is contemplated that the alkane chain may include more than 24 carbons. The FAA may also include at least one olefin in the carbon chain. The location, number, and cis or trans stereochemistry of the olefin may be varied to produce a diverse number of FAAs for use in the present invention. The FAAs may be synthesized from their corresponding fatty acids, as detailed in the '015 patent and U.S. Pat. No. 6,251,931 (“the '931 patent”), the contents of which are hereby incorporated by reference. The FAAs may also be purchased from a chemical supply company, such as Sigma-Aldrich Co. (St. Louis, Mo.). However, it is to be understood that other synthetic methods known in the art of synthesizing the FAA may be used.

The FAAH and FAA or fatty acid may be contacted with a FAAH inhibitor, which may be purchased or synthesized by techniques known in the art. For example, palmitylsulfonyl fluoride or stearylsulfonyl fluoride may be synthesized by reacting palmitylmagnesium bromide or stearylmagnesium bromide with sulfuryl chloride to produce palmitylsulfonyl chloride or stearylsulfonyl chloride as described in U.S. Pat. No. 5,874,459 (“the '459 patent”), the contents of which are hereby incorporated by reference. The palmitylsulfonyl chloride or stearylsulfonyl chloride is then reacted with ammonium fluoride to give palmitylsulfonyl fluoride or stearylsulfonyl fluoride.

The FAAH inhibitor 2-octyl-γ-bromoacetoacetate may be synthesized as known in the art. Phenylmethylsulfonyl fluoride and pyrazinamide may be purchased from Sigma-Aldrich Co. (St. Louis, Mo.). Trifluoromethyl ketones may be synthesized by converting the fatty acid into its corresponding acid chloride, followed by subsequent treatment with TFAA-pyridine, as described in U.S. Pat. No. 5,856,537 (“the '537 patent”), the contents of which are hereby incorporated by reference. Diazomethylarachidonyl ketone may be synthesized according to the method disclosed in Edgemond, et al.

The ability of the FAAH inhibitor to inhibit the deamidation of the FAA may be determined by monitoring the conversion from FAA to fatty acid (or from fatty acid to FAA). For example, the enzymatic reaction may be monitored by thin layer chromatography (“TLC”), as disclosed in the '015 patent. In addition, gas chromatography/mass spectroscopy (“GC/MS”) or radiolabeled assays may be used to monitor the reaction, as disclosed in the '459 patent. The reaction may also be monitored using an ion selective ammonia electrode to measure ammonia, which is produced as a result of the deamidation of the FAA to the fatty acid, as disclosed in the '537 patent. Further, the FAAH activity may be monitored in whole cell or membrane assays, as described in the '015 patent, Edgemond et al., or U.S. Pat. No. 6,688,825 (“the '825 patent”), the contents of which are hereby incorporated by reference. Other assays known in the art to monitor the deamidation of the FAA may also be used. Depending on the assay used to determine the potency of the FAAH inhibitor, the FAAH may be substantially pure or in a fraction isolated from a membrane preparation.

A known amount of the FAAH inhibitor may then be administered to a mammal. The FAAH inhibitor may be administered orally, rectally, or by parenteral routes, such as intramuscularly, intravenously, intraperitoneally, subcutaneously, nasally, or topically, depending on the formulation of the FAAH inhibitor. For example, if the FAAH inhibitor is formulated as a capsule or tablet, it may be administered orally or rectally. Liquid formulations of the FAAH inhibitor may be used for oral, intramuscular, intravenous, or subcutaneous administration, If the FAAH inhibitor is formulated as a powder, it may be administered nasally or by inhalation.

The ability of the FAAH inhibitor to treat the neurologic or neuropsychiatric disorder in the mammal may be determined by testing the compound in an animal model, which is selected based on the disorder to be treated. In the animal model, the mammal may suffer from the neurologic or neuropsychiatric disorder or may exhibit symptoms characteristic of the neurologic or neuropsychiatric disorder.

Preferably, the FAAH inhibitor is tested in an animal model having established validity for the particular disorder. For example, if the FAAH inhibitor is tested for its analgesic effects, the effect of the FAAH inhibitor may be measured by the standard hot plate assay (or rat flick test), where the time it takes for a rat to move its tail from a heat source is measured. If the FAAH inhibitor is being tested as a potential treatment for epilepsy, a suitable animal model for testing epilepsy that is known in the art, such as the kindling model or the kainic acid model, can be utilized. The Frings seizure model may be used to determine if the FAAH inhibitor is effective for reducing seizures. Experimental parkinsonism and MPTP-treated marmosets are commonly used animal models for Parkinson's disease. For multiple sclerosis, experimental autoimmune encephalomyelitis (“EAE”) is a valid animal model. For the treatment of migraine headaches, the amygdala kindling model, retinal plasma extravasation model, or the inhibition neurogenic dural inflammation model may be appropriate animal models. Numerous animal models for testing anxiolytic activity are known in the art, such as the “passive avoidance” model, the “elevated plus-maze” test, the Geller-Seifter test, the Vogel test, the Social Interaction Test, and the head-twitch response assay.

While exemplary animal models have been discussed, it is understood that additional animal models for each neurologic or neuropsychiatric disorder may be used within the scope of the invention. These animal models may also include transgenic animal models for the particular neurologic or neuropsychiatric disorder. Finally, human clinical trials may also be used to determine the ability of the FAAH inhibitor to treat the particular disorder in humans, once the safety and efficacy of that FAAH inhibitor has been determined in acceptable animal models.

In another specific embodiment, the present invention relates to a method for identifying fatty acids or FAAs that are useful in the treatment of neurologic or neuropsychiatric disorders. These novel FAAs or fatty acids may be resistant to hydrolysis and may retain their biological activity for extended periods. The method comprises providing a FAAH and at least one FAA or fatty acid. The FAAH FAA, or fatty acid of this embodiment may be obtained or synthesized as previously described. The ability of the FAAH to hydrolyze (or deamidate) the FAA to its corresponding fatty acid may be determined by monitoring the reaction as previously described. If the FAAH is not able to hydrolyze substantially all of a particular FAA into its corresponding fatty acid, it may indicate that the FAA or fatty acid is resistant to hydrolysis. Such an FAA or fatty acid may be useful in treating neurologic or neuropsychiatric disorders because the FAA or fatty acid would not be metabolized by the body.

A known amount of the fatty acid amide or fatty acid that is resistant to hydrolysis may be administered to a mammal as previously described. The ability of the administered fatty acid amide or fatty acid to treat the neurologic or neuropsychiatric disorder in the mammal may then be determined by observing the effects of the compound in an animal model selected for the particular disorder to be treated, as previously described.

Another embodiment of the invention relates to pharmaceutical compositions of any biologically or pharmaceutically active fatty acid(s), FAA(s), FAAH inhibitors, or derivatives thereof that are disclosed in the aforementioned methods for identifying such compounds. Pharmaceutical compositions of fatty acids, FAAs and FAAH inhibitors, or derivatives of any of these compounds may alternatively be selected from those biologically or pharmaceutically active compounds that are derived from the administration of the KD. Alternatively, precursors of biologically active fatty acid(s), FAA(s), and FAAH inhibitors may be administered to a mammal, which bypasses the need to manage a diet to produce these bioactive compounds and provides a more controlled source of the biologically active compounds.

A particular embodiment of the invention relates to a method of identifying novel FAAHs by administering isovaleramide to a mammal and monitoring the activity of FAAHs and products resulting from the metabolism of the administered isovaleramide. Alternatively, the method may be used to identify novel fatty acids, fatty acid amides, or fatty acid amide hydrolase inhibitors. The method comprises administering isovaleramide to a mammal and monitoring the activity of the fatty acid amide hydrolase and products resulting from the metabolism of the administered isovaleramide.

Alternatively, FAAHs may be employed to screen for novel FAAH inhibitors or to develop novel FAAs that are more resistant to hydrolysis and which retain their biological activity for extended periods. Alternatively, a method of identifying novel, biologically active fatty acids and FAAs includes administering a fatty acid diet (e.g., ketogenic diet) to a mammal, identifying novel fatty acids and FAAs resulting from the metabolism of the administered dietary fatty acids, and assaying the measuring the ability of the resulting fatty acids and FAAs to treat neurologic or neuropsychiatric disorders or diseases. In one particular aspect of the method, the resulting fatty acids and FAAs are altered by contacting the same with FAAH inhibitors to produce novel fatty acids and FAAs. In an alternative embodiment, novel diets of selected fatty acids and/or FAAs may be formulated for administration to mammals in order to treat neurologic or neuropsychiatric disorders or diseases.

The invention also includes a method of making pharmaceutical dosage forms containing a FAAH inhibitor for use in treating or preventing neurologic or neuropsychiatric disorders. While these compounds will typically be used in therapy for human patients, they may also be used to treat similar or identical diseases in other vertebrates such as other primates, farm animals such as swine, cattle and poultry, and sports animals and pets such as horses, dogs and cats. In therapeutic and/or diagnostic applications, the compounds of the invention may be formulated for a variety of modes of administration, including systemic, topical, or localized administration. Techniques and formulations generally may be found in Remington's Pharmaceutical Sciences, Mack Publishing Co., Easton, Pa. (18th ed., 1990).

Pharmaceutically acceptable salts are generally well known to those of ordinary skill in the art, and may be found in Remington's Pharmaceutical Sciences. The useful compounds of this invention may also be in the form of pharmaceutically acceptable complexes. Pharmaceutically acceptable complexes are known to those of ordinary skill in the art and include, by way of example but not limitation, 8-chlorotheophyllinate (teoclate). Depending on the specific conditions being treated, such agents may be formulated into liquid or solid dosage forms and administered systemically or locally. Suitable routes may include oral, buccal, sublingual, transmucosal, nasal or intestinal administration; intraperitoneal, or intranasal, just to name a few.

The compounds may be formulated readily using pharmaceutically acceptable carriers well known in the art into dosage forms suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which may be used pharmaceutically. The preparations formulated for oral administration may be in the form of tablets, dragees, capsules, or solutions.

Pharmaceutical preparations for oral use may be obtained by combining the active compounds with solid excipients, optionally grinding a resulting mixture, and processing the mixture of granules, after adding suitable auxiliaries, if desired, to obtain tablets or dragee cores. Formulations to be used according to the method of the present invention may also be incorporated into a pharmaceutical dosage form in order to protect the FAAH inhibitors from unwanted biodegradation processes or to create a sustained-release effect. Administration of pH-sensitive FAAH inhibitors may be accomplished by employing several methods to reduce acid hydrolysis in the stomach. These methods include the use of enteric coating, microencapsulation, lipid encapsulation, or administration of a buffer agent (e.g., with antacids) prior to, or concomitantly with, the administration of a formulation containing the FAA inhibitors to protect the FAA inhibitors from acids and enzymes created in portions of the gastrointestinal tract.

The invention will now be further illustrated by, but not limited to, the following examples.

### EXAMPLES

### Example 1

### Identifying an Inhibitor of Oleamide Hydrolase to Treat Epilepsy

To identify an inhibitor of oleamide hydrolase that is useful in treating epilepsy, oleamide hydrolase is added to oleamide or oleic acid. The oleamide or oleic acid is purchased or synthesized, as previously described. As previously described, the oleamide hydrolase is purchased or cloned and recombinantly expressed.

Numerous potential FAAH inhibitors, such as palmitylsulfonyl fluoride, stearylsulfonyl fluoride, 2-octyl-γ-bromoacetoacetate, phenylmethylsulfonyl fluoride, trifluoromethyl ketones, diazomethylarachidonyl ketone, and pyrazinamide, are purchased or synthesized, as previously described. Each of the potential FAAH inhibitors is separately added to a mixture of oleamide hydrolase and oleamide or oleic acid. For example, palmitylsulfonyl fluoride is added to a mixture of oleamide hydrolase and oleamide or oleic acid. The enzymatic reaction is allowed to proceed for a predetermined amount of time. Then, the ability of the oleamide hydrolase to convert the oleamide to oleic acid in the presence of palmitylsulfonyl fluoride is monitored and determined by the method described in Edgemond et al., as previously discussed. If palmitylsulfonyl fluoride is active as a FAAH inhibitor, the conversion from oleamide to oleic acid is inhibited or reduced in comparison to an enzymatic reaction that does not include palmitylsulfonyl fluoride. If palmitylsulfonyl fluoride is not active as a FAAH inhibitor, no such inhibition is achieved. This process is repeated for each potential FAAH inhibitor to determine which compounds are potent FAAH inhibitors. This screening assay is available from Novascreen Biosciences Corp. (Hanover, Md.).

A known amount of a compound active as a FAAH inhibitor is tested in an animal model for epilepsy (e.g., the kindling model). The compound is administered to a rat suffering from or exhibiting symptoms of epilepsy. The compound is administered to the rat through an appropriate route based on its formulation. If the compound is formulated as a capsule or tablet, it is administered orally or rectally. If the compound is a liquid formulation, it may be administered orally, intramuscularly, intravenously, intraperitoneally, or subcutaneously.

The ability of the compound to treat epilepsy in the affected rat is then measured. If the compound is useful to treat epilepsy, reduced seizures are observed in the affected rat. If the compound is not useful to treat epilepsy, the seizures are not reduced.

The compound having FAAH inhibitor activity is also administered to the rat in conjunction with a fatty acid amide or a fatty acid. The ability of the compound, in combination with the fatty acid amide or the fatty acid, to treat epilepsy in the affected rat is measured. If the compound and fatty acid amide or fatty acid are useful in treating epilepsy, reduced seizures are observed in the affected rat.

### Example 2

### Identifying an Inhibitor of Anandamide Amidase to Treat Epilepsy

To identify an inhibitor of anandamide amidase that is useful in treating epilepsy, the method described in Example 1 is followed, except that anandamide amidase is used instead of oleamide hydrolase. As previously described herein, the anandamide amidase is purchased or cloned and recombinantly expressed.

### Example 3

### Identifying a FAA or Fatty Acid to Treat Epilepsy

To identify a novel FAA or fatty acid useful in the treatment of epilepsy, oleamide hydrolase is added to at least one FAA or fatty acid. The oleamide hydrolase is obtained as previously described. The FAA or fatty acid is purchased or synthesized as previously described. Numerous FAAs or fatty acids having diverse structures are tested. The enzymatic reaction is allowed to proceed for a predetermined amount of time. The ability of the oleamide hydrolase to deamidate or hydrolyze the FAA to its corresponding fatty acid is determined by monitoring the reaction as disclosed in the '015 patent. If the FAA is not substantially deamidated by the oleamide hydrolase, it indicates that the FAA or fatty acid is resistant to hydrolysis by the oleamide hydrolase.

A known amount of the fatty acid amide or fatty acid that is resistant to hydrolysis is tested in the kindling model. The fatty acid amide or fatty acid is administered to a rat suffering from or exhibiting symptoms of epilepsy. As described with reference to Example 1, the fatty acid amide or fatty acid is administered through an appropriate route based on its formulation.

The ability of the administered fatty acid amide or fatty acid to treat epilepsy in the affected rat is measured by observing the effects of the fatty acid amide or fatty acid in the kindling model. If the fatty acid amide or fatty acid is useful to treat epilepsy, reduced seizures are observed in the affected rat and the compound(s) responsible for such reduction are identified as therapeutic agent(s). If the fatty acid amide or fatty acid is not useful to treat epilepsy, seizures are not reduced and such compounds are eliminated as potential drug candidates.

### Example 4

### Identifying a FAA or Fatty Acid to Treat Epilepsy

To identify a novel FAA or fat acid useful in the treatment of epilepsy, the method described in Example 3 is followed, except that anandamide amidase is used instead of oleamide hydrolase. The anandamide amidase is obtained as previously described herein.

### Example 5

### Identifying an Inhibitor of Oleamide Hydrolase to Treat Anxiety Disorders

To identify an inhibitor of oleamide hydrolase that is useful in treating anxiety disorders, the method described in Example 1 is followed, with the following changes. A known amount of a compound active as a FAAH inhibitor is tested in the elevated plus-maze test, a known animal model for anxiety. The compound is administered to a rat suffering from anxiety. The compound is administered to the rat through an appropriate route based on its formulation. If the compound is formulated as a capsule or tablet, it is administered orally or rectally. If the compound is a liquid formulation, it is administered orally, intramuscularly, intravenously, intraperitoneally, or subcutaneously.

The ability of the compound to treat anxiety in the rat is measured. If the compound is effective in treating anxiety, symptoms associated with anxiety are reduced or ameliorated. If the compound is not effective in treating anxiety, the symptoms associated with anxiety are not reduced.

The compound active as a FAAH inhibitor is also administered to the rat in conjunction with a fatty acid amide or fatty acid. The ability of the compound, in combination with the fatty acid amide or fatty acid, to treat anxiety in the affected rat is measured. If the compound and fatty acid amide or fatty acid are useful to treat anxiety, the symptoms associated with anxiety are reduced in the rat and the compound(s) responsible for such reduction are identified as therapeutic agent(s).

### Example 6

### Identifying an Inhibitor of Anandamide Amidase to Treat an Anxiety Disorder

To identify an inhibitor of anandamide amidase that is useful in treating an anxiety disorder, the method described in Example 5 is followed, except that anandamide amidase is used instead of oleamide hydrolase. The anandamide amidase may be purchased or may be cloned and recombinantly expressed.

### Example 7

### Identifying a FAA or Fatty Acid to Treat an Anxiety Disorder

To identify a novel FAA or fatty acid useful in the treatment of an anxiety disorder, the method of Example 3 is followed, except that the known amount of fatty acid amide or fatty acid that is resistant to hydrolysis is tested in the elevated plus-maze test, a known animal model for anxiety. The fatty acid amide or fatty acid is administered to a rat suffering from anxiety. As previously discussed, the fatty acid amide or fatty acid is administered through an appropriate route based on its formulation.

The ability of the administered fatty acid amide or fatty acid to treat anxiety in the rat is measured by observing the effects of the fatty acid amide or fatty acid on the rat. If the fatty acid amide or fatty acid is useful to treat anxiety, the symptoms associated with anxiety are reduced or ameliorated in the rat and the compound(s) responsible for such reduction are identified as therapeutic agent(s).

### Example 8

### Identifying a FAA or Fatty Acid to Treat an Anxiety Disorder

To identify a novel FAA or fatty acid useful in the treatment of an anxiety disorder, the method of Example 7 is followed, except that anandamide amidase is used instead of oleamide hydrolase. The anandamide amidase may be obtained as previously described herein.

### Example 9

### Identifying an Inhibitor of Oleamide Hydrolase to Treat Parkinson's Disease

To identify an inhibitor of oleamide hydrolase that is useful in treating Parkinson's disease, the method described in Example 1 is followed with the following changes. A known amount of a compound active as a FAAH inhibitor is tested in MPTP-treated marmosets, a known animal model for Parkinson's disease. The compound is administered to marmosets suffering from Parkinson-like symptoms. The compound is administered to the marmoset through an appropriate route based on its formulation.

The ability of the compound to treat the symptoms associated with Parkinson's disease is measured. The compound is effective in treating Parkinson's disease if one or more symptoms associated with the disease, such as rigidity, akinesia, and tremors, are reduced or ameliorated. If the symptoms are not reduced, the compound is not considered effective in treating the disease.

The compound active as a FAAH inhibitor is also administered to the marmoset in conjunction with a fatty acid amide or fatty acid. The ability of the compound, in combination with the fatty acid amide or fatty acid, to treat Parkinson-like symptoms in the marmoset is measured. If the compound and fatty acid amide or fatty acid are useful to treat Parkinson's disease, the symptoms associated with the disease are reduced and the compound(s) responsible for such reduction are identified as therapeutic agent(s).

### Example 10

### Identifying an Inhibitor of Anandamide Amidase to Treat Parkinson's Disease

To identify an inhibitor of anandamide amidase that is useful in treating Parkinson's disease, the method described in Example 9 is followed, except that anandamide amidase is used instead of oleamide hydrolase. As previously described, the anandamide amidase may be purchased or may be cloned and recombinantly expressed.

### Example 11

### Identifying a FAA or Fatty Acid to Treat Parkinson's Disease

To identify a novel FAA or fatty acid useful in the treatment of Parkinson's disease, the method of Example 3 is followed except that the known amount of fatty acid amide or fatty acid that is resistant to hydrolysis is tested in MPTP-treated marmosets. The fatty acid amide or fatty acid is administered to a marmoset suffering from Parkinson-like symptoms. The fatty acid amide or fatty acid is administered based on its formulation. If the fatty acid amide or fatty acid is formulated as a capsule or tablet, it is administered orally or rectally. If the fatty acid amide or fatty acid is a liquid formulation, it is administered orally, intramuscularly, intravenously, intraperitoneally, or subcutaneously.

The ability of the administered fatty acid amide or fatty acid to treat the Parkinson-like symptoms in the affected marmoset is measured by observing the effects of the fatty acid amide or fatty acid on the MPTP-treated marmosets. If one or more Parkinson-like symptoms are reduced or ameliorated in the marmoset, the fatty acid amide or fatty acid is useful to treat Parkinson's disease and the compound(s) responsible for such reduction are identified as therapeutic agent(s).

### Example 12

### Identifying a FAA or Fatty Acid to Treat Parkinson's Disease

To identify a novel FAA or fatty acid useful in the treatment of Parkinson's disease, the method described in Example 11 is followed, except that anandamide amidase is used instead of oleamide hydrolase. The anandamide amidase is obtained as previously described herein.

### Example 13

### Identifying an Inhibitor of Oleamide Hydrolase Useful as an Analgesic

To identify an inhibitor of oleamide hydrolase that is useful as an analgesic, the method described in Example 1 is followed, with the following changes. A known amount of a compound active as a FAAH inhibitor is tested in the standard hot plate assay, a known animal model for determining the analgesic effects of the compound. The compound is administered to a rat based on its formulation. If the compound is formulated as a capsule or tablet, it is administered orally or rectally. If the compound is a liquid formulation, it is administered orally, intramuscularly, intravenously, intraperitoneally, or subcutaneously. If the compound is effective as an analgesic, the amount of time it takes for the rat to move its tail away from a heat source is increased relative to a rat that did not receive the compound.

The compound active as a FAAH inhibitor is also administered to the rat in conjunction with a fatty acid amide or fatty acid. If the combination of the compound and fatty acid amide or fatty acid is effective as an analgesic, the amount of time it takes for the rat to move its tail away from the heat source is increased relative to the rat that did not receive the compound and fatty acid amide or fatty acid. Effective compound(s) are identified as possible therapeutic agent(s).

### Example 14

### Identifying an Inhibitor of Anandamide Amidase Useful as an Analgesic

To identify an inhibitor of anandamide amidase that is useful as an analgesic, the method described in Example 13 is followed, except that anandamide amidase is used instead of oleamide hydrolase. As previously described herein, the anandamide amidase may be purchased or may be cloned and recombinantly expressed.

### Example 15

### Identifying a FAA or Fatty Acid Useful as an Analgesic

To identify a novel FAA or fatty acid useful as an analgesic, the method of Example 3 is followed except that the known amount of fatty acid amide or fatty acid that is resistant to hydrolysis is tested in the standard hot plate assay. The compound is administered to a rat based on its formulation. If the compound is formulated as a capsule or tablet, it is administered orally or rectally. If the compound is a liquid formulation, it is administered orally, intramuscularly, intravenously, intraperitoneally, or subcutaneously. If the compound is effective as an analgesic, the amount of time it takes for the rat to move its tail away from the heat source is increased relative to the rat that did not receive the compound.

### Example 16

### Identifying a FAA or Fatty Acid Useful as an Analgesic

To identify a novel FAA or fatty acid useful as an analgesic, the method described in Example 15 is followed, except that anandamide amidase is used instead of oleamide hydrolase. The anandamide amidase is obtained as previously described herein.

### Example 17

### Identifying an Inhibitor of Oleamide Hydrolase to Treat Multiple Sclerosis

To identify an inhibitor of oleamide hydrolase that is useful in treating multiple sclerosis, the method described in Example 1 is followed, with the following changes. A known amount of a compound active as a FAAH inhibitor is tested in the EAE model, a known animal model for multiple sclerosis. The compound is administered to a rat showing symptoms of multiple sclerosis. The compound is administered to the rat based on its formulation. If the compound is formulated as a capsule or tablet, it is administered orally or rectally. If the compound is a liquid formulation, it is administered orally, intramuscularly, intravenously, intraperitoneally, or subcutaneously.

The ability of the compound to treat the multiple sclerosis-like symptoms in the rat is measured. If the compound is effective in treating multiple sclerosis, one or more symptoms associated with multiple sclerosis are reduced or ameliorated.

The compound active as a FAAH inhibitor is also administered to the rat in conjunction with a fatty acid amide or fatty acid. The ability of the compound, in combination with the fatty acid amide or fatty acid, to treat the symptoms of multiple sclerosis in the affected rat is measured. If one or more symptoms are reduced, the combination of the compound and fatty acid amide or fatty acid is useful to treat multiple sclerosis.

### Example 18

### Identifying an Inhibitor of Anandamide Amidase to Treat Multiple Sclerosis

To identify an inhibitor of anandamide amidase that is useful in the treatment of multiple sclerosis, the method described in Example 17 is followed, except that anandamide amidase is used instead of oleamide hydrolase. As previously described herein, the anandamide amidase may be purchased or may be cloned and recombinantly expressed.

### Example 19

### Identifying a FAA or Fatty Acid to Treat Multiple Sclerosis

To identify a novel FAA or fatty acid useful in the treatment of multiple sclerosis, the method of Example 3 is followed except that the known amount of fatty acid amide or fatty acid that is resistant to hydrolysis is tested in the EAE model. The fatty acid amide or fatty acid is administered to a rat having multiple sclerosis-like symptoms. The fatty acid amide or fatty acid is administered based on its formulation. If the fatty acid amide or fatty acid is formulated as a capsule or tablet, it is administered orally or rectally. If the fatty acid amide or fatty acid is a liquid formulation, it is administered orally, intramuscularly, intravenously, intraperitoneally, or subcutaneously.

The ability of the administered fatty acid amide or fatty acid to treat one or more multiple sclerosis-like symptoms is measured by observing the effects of the fatty acid amide or fatty acid on the rat. If the multiple sclerosis-like symptoms are reduced or ameliorated in the rat, the fatty acid amide or fatty acid is identified as being useful to treat multiple sclerosis.

### Example 20

### Identifying a FAA or Fatty Acid to Treat Multiple Sclerosis

To identify a novel FAA or fatty acid useful in the treatment of multiple sclerosis, the method described in Example 19 is followed, except that anandamide amidase is used instead of oleamide hydrolase. The anandamide amidase is obtained as previously described herein.

### Example 21

### Identifying an Inhibitor of Oleamide Hydrolase to Treat a Migraine Headache

To identify an inhibitor of oleamide hydrolase that is useful in treating a migraine headache, the method described in Example 1 is followed, with the following changes. A known amount of a compound active as a FAAH inhibitor is tested in the neurogenic dural inflammation model, which is predictive of compounds that may be effective therapies for migraine headaches. The compound is administered to a rat showing the symptoms of a migraine headache. The compound is administered to the rat based on its formulation. If the compound is formulated as a capsule or tablet, it is administered orally or rectally. If the compound is a liquid formulation, it is administered orally, intramuscularly, intravenously, intraperitoneally, or subcutaneously.

The ability of the compound to treat the symptoms of the migraine headache (or inhibit neurogenic inflammation) in the rat is measured. If the compound is effective, the neurogenic inflammation is reduced or ameliorated.

The compound active as a FAAH inhibitor is also administered to a rat in conjunction with a fatty acid amide or fatty acid. The ability of the compound, in combination with the fatty acid amide or fatty acid, to treat the symptoms of the migraine headache (or inhibit neurogenic inflammation) is measured. If the neurogenic inflammation is reduced, the combination of the compound and fatty acid amide or fatty acid is identified as being useful to treat the migraine headache.

### Example 22

### Identifying an Inhibitor of Anandamide Amidase to Treat a Migraine Headache

To identify an inhibitor of anandamide amidase that is useful in the treatment of a migraine headache, the method described in Example 21 is followed, except that anandamide amidase is used instead of oleamide hydrolase. As previously described herein, the anandamide amidase is purchased or cloned and recombinantly expressed.

### Example 23

### Identifying a FAA or Fatty Acid to Treat a Migraine Headache

To identify a novel FAA or fatty acid useful in the treatment of a migraine headache, the method of Example 3 is followed except that the known amount of fatty acid amide or fatty acid that is resistant to hydrolysis is tested in the neurogenic dural inflammation model. The fatty acid amide or fatty acid is administered to a rat having symptoms of a migraine headache. The fatty acid amide or fatty acid is administered through an appropriate route based on its formulation. If the fatty acid amide or fatty acid is formulated as a capsule or tablet, it is administered orally or rectally. If the fatty acid amide or fatty acid is a liquid formulation, it is administered orally, intramuscularly, intravenously, intraperitoneally, or subcutaneously.

The ability of the administered fatty acid amide or fatty acid to treat the symptoms of the migraine (or inhibit neurogenic inflammation) is measured by observing the effects of the fatty acid amide or fatty acid on the rat. If the neurogenic inflammation is reduced or ameliorated in the rat, the fatty acid amide or fatty acid is useful to treat the disorder.

### Example 24

### Identifying a FAA or Fatty Acid to Treat a Migraine Headache

To identify a novel FAA or fatty acid useful in the treatment of a migraine headache, the method described in Example 23 is followed, except that anandamide amidase is used instead of oleamide hydrolase. The anandamide amidase is obtained as previously described herein.

What is claimed is:

1. A method of treating a neurologic or neuropsychiatric disorder in a mammal, said method comprising:
administering to said mammal a fatty acid amide hydrolase inhibitor in an amount sufficient to inhibit deamidation of a fatty acid amide, wherein said neurologic or neuropsychiatric disorder is selected from the group consisting of a mood disorder, a migraine headache, a spastic condition, a restless limb syndrome, a movement disorder, anxiety, epilepsy, or a neurodegenerative disease.

2. The method of claim 1, wherein said mammal is a human.

3. The method of claim 1, further comprising administering to said mammal a fatty acid amide or a fatty acid

4. The method of claim 1, wherein said neurologic or neuropsychiatric disorder is epilepsy.

5. The method of claim 1, wherein administering to said mammal a fatty acid amide hydrolase inhibitor comprises administering an inhibitor selected from the group consisting of palmitylsulfonyl fluoride, stearylsulfonyl fluoride, 2-octyl-γ-bromoacetoacetate, phenylmethylsulfonyl fluoride, trifluoromethyl ketones, diazomethylarachidonyl ketone, and pyrazinamide.

6. The method of claim 1, wherein said fatty acid amide hydrolase inhibitor inhibits the deamidation of anandamide.

7. The method of claim 1, wherein said fatty acid amide hydrolase inhibitor inhibits the deamidation of oleamide.

8. The method of claim 1, wherein said fatty acid amide hydrolase inhibitor inhibits the deamidation of a fatty acid amide that modulates serotonergic neurotransmissions.

9. The method of claim 1, wherein said fatty acid amide hydrolase inhibitor is administered in an amount sufficient to inhibit deamidation of exogenously administered fatty acid amides.

10. The method of claim 1, wherein said fatty acid amide hydrolase inhibitor is administered in an amount sufficient to inhibit deamidation of endogenous fatty acid amides.

11. The method of claim 1, further comprising administering to said mammal an anticonvulsant.

12. The method of claim 1, further comprising administering to said mammal at least one ketogenic amino acid.

13. A method of treating a sleep disorder in a mammal comprising:
administering to said mammal a fatty acid amide hydrolase inhibitor in an amount sufficient to inhibit deamidation of a fatty acid amide, wherein said fatty acid amide hydrolase inhibitor is selected from the group consisting of palmitylsulfonyl fluoride, stearylsulfonyl fluoride, phenylmethylsulfonyl fluoride, diazomethylarachidonyl ketone, and pyrazinamide.

14. A method of treating a pain disorder in a mammal comprising:
administering to said mammal a fatty acid amide hydrolase inhibitor in an amount sufficient to inhibit deamidation of a fatty acid amide, wherein said fatty acid amide hydrolase inhibitor is selected from the group consisting of stearylsulfonyl fluoride, phenylmethylsulfonyl fluoride, trifluoromethyl ketones, diazomethylarachidonyl ketone, and pyrazinamide.

15. A method for identifying a fatty acid amide hydrolase inhibitor useful in the treatment of a neurologic or neuropsychiatric disorder, the method comprising:
providing a fatty acid amide hydrolase and at least one fatty acid amide or fatty acid;
adding said fatty acid amide hydrolase inhibitor said at least one fatty acid amide or fatty acid;
determining the ability of said fatty acid amide hydrolase inhibitor to inhibit the conversion between said at least one fatty acid amide and its corresponding fatty acid or between said at least one fatty acid and its corresponding fatty acid amide;
administering a known amount of said fatty acid amide hydrolase inhibitor to a mammal; and
determining the ability of said fatty acid amide hydrolase inhibitor to treat said neurologic or neuropsychiatric disorder in said mammal.

16. The method of claim 15, wherein said fatty acid amide hydrolase inhibitor is an inhibitor selected from the group consisting of palmitylsulfonyl fluoride, stearylsulfonyl fluoride, 2-octyl-γ-bromoacetoacetate, phenylmethylsulfonyl fluoride, trifluoromethyl ketones, diazomethylarachidonyl ketone, and pyrazinamide.

17. The method of claim 15, wherein said fatty acid amide hydrolase inhibitor inhibits the deamidation of anandamide.

18. The method of claim 15, wherein said fatty acid amide hydrolase inhibitor inhibits the deamidation of oleamide.

19. The method of claim 15, wherein said fatty acid amide hydrolase inhibitor inhibits the deamidation of a fatty acid amide that modulates serotonergic neurotransmissions.

20. The method of claim 15, wherein said fatty acid amide is synthetic.

21. The method of claim 15, wherein said mammal is a human.

22. The method of claim 15, wherein said neurologic or neuropsychiatric disorder is selected from the group consisting of epilepsy, pain, a sleep disorder, anxiety, a mood disorder, a migraine headache, a spastic neurodegenerative disease, and a movement disorder.

23. The method of claim 15, further comprising administering to said mammal a known amount of a fatty acid amide.

24. The method of claim 15, further comprising administering to said mammal a known amount of a fatty acid.

25. A method for identifying a fatty acid amide or fatty acid useful in the treatment of a neurologic or neuropsychiatric disorder, the method comprising:
providing a fatty acid amide hydrolase and at least one fatty acid amide or fatty acid;
determining the ability of said fatty acid amide hydrolase to convert said at least one fatty acid amide to its corresponding fatty acid or said at least one fatty acid to its corresponding fatty acid amide to identify a fatty acid amide or fatty acid that is resistant to hydrolysis;
administering to a mammal a known amount of said fatty acid amide or fatty acid that is resistant to hydrolysis; and
determining the ability of said fatty acid amide or fatty acid that is resistant to hydrolysis to treat said neurologic or neuropsychiatric disorder in said mammal.

26. The method of claim 25, wherein said fatty acid amide hydrolase inhibitor is an inhibitor selected from the group consisting of palmitylsulfonyl fluoride, stearylsulfonyl fluoride, 2-octyl-γ-bromoacetoacetate, phenylmethylsulfonyl fluoride, trifluoromethyl ketones, diazomethylarachidonyl ketone, and pyrazinamide.

27. The method of claim 25, wherein said neurologic or neuropsychiatric disorder is selected from the group consisting of epilepsy, pain, a sleep disorder, anxiety, a mood disorder, a migraine headache, a spastic condition, a restless limb syndrome, a neurodegenerative disease, and a movement disorder.

28. The method of claim 25, wherein said at least one fatty acid amide or fatty acid that is resistant to hydrolysis comprises an alkane chain of approximately six to fifteen carbons.

29. The method of claim 28, wherein said at least one fatty acid amide or fatty acid that is resistant to hydrolysis comprises a branched chain.
